(19) United States
(12) Patent Application Publication
(10) Pub. No.: US 2024/0024129 A1
(43) Pub. Date: Jan. 25, 2024

(54) APPARATUS AND METHOD FOR FASTENER ALIGNMENT OF AN ORTHOPEDIC IMPLANT

(71) Applicant: Arthrex, Inc., Naples, FL (US)

(72) Inventors: Nick Metcalfe, Bonita Springs, FL (US); Steven Jim DeLeon, Naples, FL (US)

(73) Assignee: Arthrex, Inc, Naples, FL (US)

(21) Appl. No.: 18/223,612

(22) Filed: Jul. 19, 2023

Related U.S. Application Data
(60) Provisional application No. 63/391,083, filed on Jul. 21, 2022.

(51) Int. Cl. A61F 2/46; A61B 17/17

(52) U.S. Cl. CPC A61F 2/4612; A61B 17/1778

(57) ABSTRACT

An alignment apparatus for an orthopedic implant includes a coupling fixture including a plurality of alignment features that align with and engage complementary features of the orthopedic implant. The alignment features orient an alignment axis of the alignment apparatus with at least one fastener aperture of the orthopedic implant. A guide insert includes a guide aperture through which the alignment axis passes. The guide aperture is configured to selectively align a tool or fastener with a plurality of trajectories through the at least one fastener aperture.

### CROSS-REFERENCE TO RELATED APPLICATIONS

This application claims priority under 35 U.S.C. § 119(e) and the benefit of U.S. Provisional Application No. 63/391,083 entitled APPARATUS AND METHOD FOR FASTENER ALIGNMENT OF AN ORTHOPEDIC IMPLANT, filed on Jul. 21, 2022, by Metcalfe et al., the entire disclosure of which is incorporated herein by reference.

### BACKGROUND

The present disclosure generally relates to an apparatus and method for an orthopedic surgical operation. More particularly, the disclosure may provide for an alignment apparatus for aligning at least one fastener of an orthopedic implant along a trajectory for connection with the anatomy of a patient. The apparatus and corresponding methods may provide for accurate alignment of the at least one fastener while also providing flexibility in adjusting the trajectory of the fastener during a surgical procedure.

### SUMMARY

The disclosure generally provides for an apparatus and associated methods to improve the engagement of an orthopedic implant with an anatomy of a patient. In general, in order to securely fasten an implant to the corresponding anatomy of a patient, each fastener must be aligned along a specific trajectory, such that the fastener engages designated structural regions of bone or tissue of the patient. In order to identify alignment trajectories for fasteners of an orthopedic implant, advance planning and simulation software may be implemented to model the anatomy of the patient and accurately identify the associated trajectory angles for the fasteners. However, despite the accuracy of such models, surgical plans, and trajectory or alignment plans; anatomic fasteners may require adjustments during a surgical procedure. Such adjustments may be significantly limited by some patient-specific, trajectory alignment devices associated with specific surgical plans. In various implementations, the disclosure provides for an alignment apparatus and methods that not only provide for the accurate alignment of fasteners and pilot holes along specified or predefined trajectories but also provide for the adjustment of such trajectory angles by surgeons during orthopedic procedures.

As further discussed in the following detailed description, the alignment apparatus may provide for the alignment of a plurality of fasteners at complex or compound angles defined relative to a portion or base plate of an implant. For example, a coupling fixture of the alignment apparatus may engage the base plate and orient an alignment axis central to a fastener aperture of the base plate. In such implementations, the alignment apparatus may provide intuitive alignment features that engage complementary features of the implant. With the coupling fixture engaged with the base plate, the trajectories of the fasteners may be easily and accurately adjusted along complex trajectories during an operation. Accordingly, the disclosed apparatus may provide for flexible adjustment of the alignment trajectories for a plurality of fasteners while limiting the associated complexity. In this way, the device may provide for improved methods for securing an orthopedic implant by achieving decreased operating times and improved patient outcomes.

In some implementations, the alignment features of the alignment apparatus may orient the alignment axis of the alignment apparatus with at least one fastener aperture of an orthopedic implant. Additionally, a guide insert may engage the coupling fixture and provide a guide aperture through which the alignment axis of the coupling fixture passes. The guide aperture may be configured to selectively align a tool (e.g., a cutting tool, drill, driver, etc.) or fastener (e.g., a screw, pin, etc.) at a plurality of trajectories or trajectory angles through the at least one fastener aperture of the orthopedic implant. As presented in various examples, the implant may engage a patient via a plurality of implant fasteners. In such cases, the alignment features of the alignment apparatus may engage the complementary features of the base plate of the orthopedic implant in a plurality of rotational orientations. In this way, the alignment apparatus may be arranged to selectively align the alignment axis with each of the plurality of fastener apertures.

In various implementations, the disclosure may further provide for one or more of the following features or steps alone or in combination:
at least one fastener aperture of the implant comprises a plurality of fastener apertures, and wherein the alignment features engage the complementary features in a plurality of rotational orientations relative to the orthopedic implant;
the plurality of rotational orientations selectively align the alignment axis of the coupling fixture with each of the plurality of fastener apertures;
the coupling fixture comprises a collar that receives the orthopedic implant and aligns a central axis of the implant with the alignment features;
the alignment features comprise a plurality of alignment pins that engage a plurality of coupling apertures of the orthopedic implant;
the coupling fixture aligns the alignment axis at a first angle relative to a mounting surface of the orthopedic implant;
the first angle is between 1° and 45°;
the guide insert engages the coupling fixture along a guide plane defined by a first angle and perpendicular to the alignment axis;
the guide insert is rotationally constrained to rotate about the alignment axis;
the guide aperture of the guide insert is angled at a second angle relative to the alignment axis.
the second angle of the plurality of guide inserts varies from 0.1° to 15°;
the first angle and the second angle define a compound angle defining a path of the trajectories through the at least one fastener aperture;
a rotation of the guide insert relative to the coupling fixture selectively aligns the guide aperture with the plurality of trajectory angles of the tool or fastener;
the guide insert is an interchangeable component comprising a plurality of guide inserts, wherein the second angle of each of the plurality of guide inserts is different, such that the second angle is selected based on the guide insert selected;
the guide insert corresponds to a cylindrical dial that engages a pocket of the coupling fixture;
the coupling fixture comprises an indicator surface extending about the pocket of the coupling fixture;
the indicator surface comprises an angle indicia and the guide insert comprises an alignment indicator, and wherein a rotational angle of guide insert relative to the coupling fixture is identified by an orientation of the alignment indicator with the angle indicia; and/or
the orthopedic implant is a glenoid implant.

In some implementations, the disclosure may provide a method for aligning a fastener for an orthopedic implant. The method may include aligning an alignment axis of a coupling fixture with a first fastener aperture of a plurality of fastener apertures of the orthopedic implant. The alignment axis of the coupling fixture may aligned by engaging a plurality of alignment features with a plurality of complementary features of the orthopedic implant in a first orientation. The method may further comprise a step of removing the coupling fixture from engagement with the orthopedic implant and rotating the coupling fixture relative to the orthopedic implant. The coupling fixture may then be aligned with a second fastener aperture of the orthopedic implant by engaging the plurality of alignment features with the plurality of complementary features of the orthopedic implant in a second orientation.

In various implementations, the disclosure further provides for one or more of the following features or steps alone or in combination:
the coupling fixture engages the orthopedic implant by sliding the alignment features linearly along the complementary features of the orthopedic implant;
the rotation of the coupling fixture is about a central axis of the orthopedic implant and aligns the plurality of alignment features with the plurality of complementary features of the orthopedic implant;
the alignment features and the fastener apertures are evenly distributed about the central axis;
selectively engaging a guide insert comprising a guide aperture with the coupling fixture, wherein the engagement between the guide insert and the coupling fixture constrains a rotation of the guide insert to rotate about the alignment axis; and/or
the guide aperture is angled at a first angle relative to the alignment axis, wherein the rotation of the guide insert about the alignment axis adjusts a trajectory angle of the guide aperture relative to the alignment axis; and further comprising aligning a tool or fastener through the guide aperture at a rotational angle about the alignment axis, thereby defining a trajectory of the tool or fastener.

In some implementations an alignment apparatus for an orthopedic implant is disclosed. The apparatus may comprise a coupling fixture comprising a plurality of alignment features and alignment complementary features of the orthopedic implant. The alignment features orient in an alignment axis of the alignment apparatus with at least one fastener aperture of the orthopedic implant. A guide insert that engages the coupling fixture along a guide plane defined by a first angle perpendicular to the alignment axis. A rotation of the guide insert relative to the coupling fixture is constrained to rotate about the alignment axis in an assembled configuration. The guide insert comprises a guide aperture angled at a second angle relative to the alignment axis. The rotation of the guide insert is relative to a plurality of trajectory angles of a tool or fastener.

These and other features, objects and advantages of the present disclosure will become apparent upon reading the following description thereof together with reference to the accompanying drawings.

### BRIEF DESCRIPTION OF THE DRAWINGS

FIG. 1A is a diagram demonstrating a base plate of an orthopedic implant in connection with an exemplary model of a scapula of a patient;

FIG. 1B is an illustrative diagram demonstrating an engagement and operation of an alignment apparatus in connection with the orthopedic implant demonstrated in FIG. 1A;

FIG. 1C is a diagram demonstrating a base plate of a custom or patient-specific orthopedic implant in connection with an exemplary model of a scapula of a patient;

FIG. 1D is an illustrative diagram demonstrating an engagement and operation of an alignment apparatus in connection with the orthopedic implant demonstrated in FIG. 1C;

FIG. 2 is a projected view of a base plate of an orthopedic implant;

FIG. 3A is a projected view of an alignment apparatus for an orthopedic implant;

FIG. 3B is a side view of an alignment apparatus for an orthopedic implant;

FIG. 3C is a top view of an alignment apparatus for an orthopedic implant;

FIG. 3D is a bottom view of an alignment apparatus for an orthopedic implant;

FIG. 4 is an instructional diagram demonstrating a rotational alignment of alignment features of an alignment apparatus with complementary features of an orthopedic implant;

FIG. 5A is a projected view of an interchangeable guide insert aligned with an alignment axis of a coupling fixture of an alignment apparatus;

FIG. 5B is a projected view of an interchangeable guide insert demonstrating a guide aperture angled at a first angle relative to an alignment axis of an alignment apparatus;

FIG. 5C is a projected view of an interchangeable guide insert demonstrating a guide aperture angled at a second angle relative to an alignment axis of an alignment apparatus;

FIG. 5D is a projected view of an interchangeable guide insert demonstrating a guide aperture angled at a third angle relative to an alignment axis of an alignment apparatus;

FIG. 6 is a geometric representation of an adjustment of a trajectory angle of a tool or fastener based on a rotational adjustment of a guide insert relative to a coupling fixture of the alignment apparatus;

FIG. 7 is a projected view of a base plate of an orthopedic implant in connection with a glenoid cavity;

FIG. 8A is a projected view of a coupling fixture in connection with a base plate of an orthopedic implant;

FIG. 8B is a side view of a coupling fixture in connection with a base plate of an orthopedic implant;

FIG. 9A is a projected view of an alignment apparatus in connection with a base plate of an orthopedic implant demonstrating a rotational adjustment of a guide insert;

FIG. 9B is a side view demonstrating a cutting tool forming a pilot hole along a trajectory angle defined by the alignment apparatus; and

FIG. 9C is a side view demonstrating a driver or fastener tool driving a fastener along a trajectory angle defined by the alignment apparatus in accordance with the disclosure.

### DETAILED DESCRIPTION

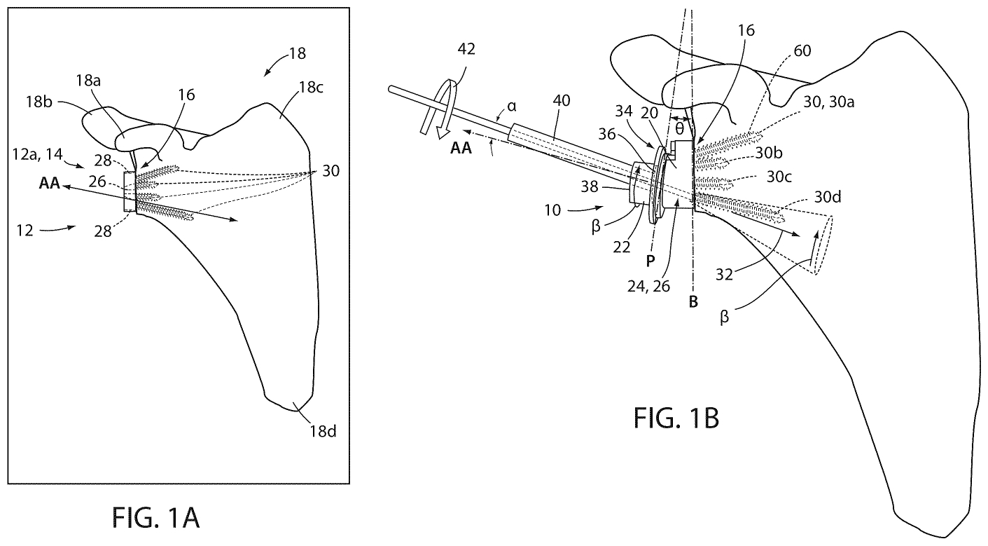

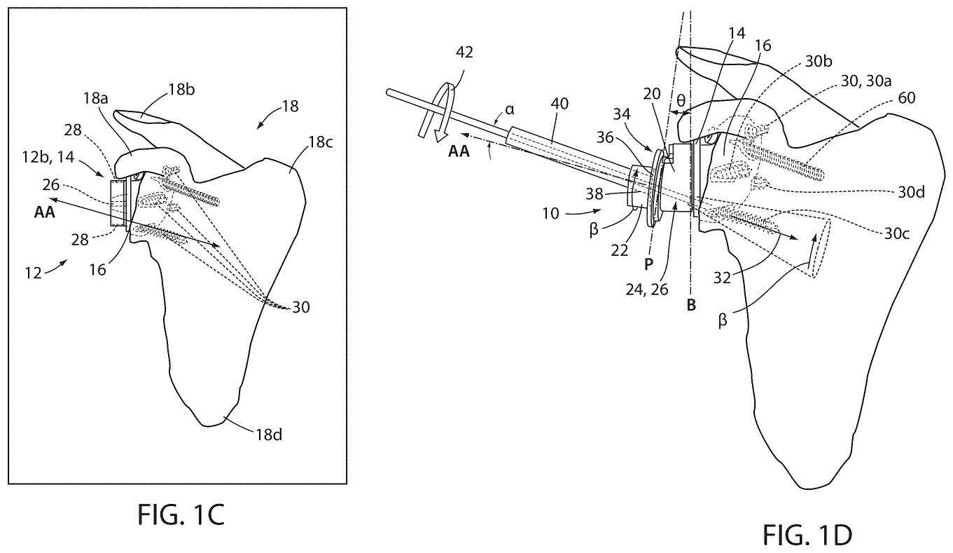

The disclosure generally provides for an alignment apparatus configured to adjustably align a tool or fastener with at least one fastener aperture of an orthopedic implant. In various implementations, the alignment apparatus may be configured to engage a plurality of alignment features of the orthopedic implant, such that an alignment axis of the alignment apparatus is aligned with the at least one fastener aperture of the orthopedic implant. As demonstrated in FIGS. 1A, 1B, 1C, and 1D, the alignment apparatus 10 is shown in reference to an exemplary orthopedic implant 12, exemplified as a glenoid implant comprising a base plate 14. The exemplary implants 12 demonstrated in FIGS. 1A-D differ in that FIGS. 1A and 1B demonstrate a standard implant 12a while FIGS. 1C and 1D demonstrate a custom or patient-specific implant 12b. The primary difference between the standard implant 12a and the custom implant 12b is that the custom implant 12b may include patient-specific features, which may be beneficial in some reconstructive applications. In either example, the alignment apparatus 10 may be implemented similarly to ensure that the implants 12a and 12b are accurately secured to the patient. In each example shown in FIGS. 1A-1D, the base plate 14 is shown in connection with a glenoid cavity 16 of a model scapula 18 of a patient. For a visual reference, the scapula 18 is shown demonstrating a coracoid process 18a, an acromion 18b, a superior angle 18c, and an inferior angle 18d.

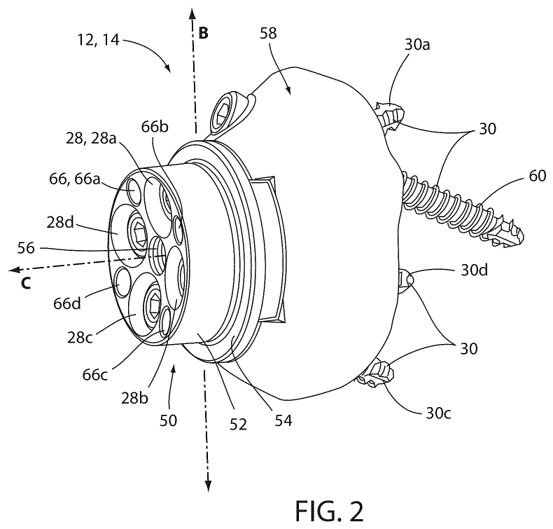

As demonstrated in FIGS. 1B and 1D, the alignment apparatus 10 may comprise a coupling fixture 20 and a guide insert 22. As further discussed in reference to FIGS. 2-4, the coupling fixture 20 may comprise a plurality of alignment features 24 configured to selectively engage complementary mating features 26 of the base plate 14 in a plurality of rotational orientations I, II, III, IV. In this way, the alignment apparatus 10 may provide for the selective positioning of an alignment axis AA with each of a plurality of fastener apertures 28 of the base plate 14. Accordingly, the alignment apparatus 10 may provide for the alignment of a plurality of fasteners 30 along specified or selected trajectory angles and corresponding trajectories 32 for effective connection of the implant 12 to the anatomy (e.g., the scapula 18) of a patient.

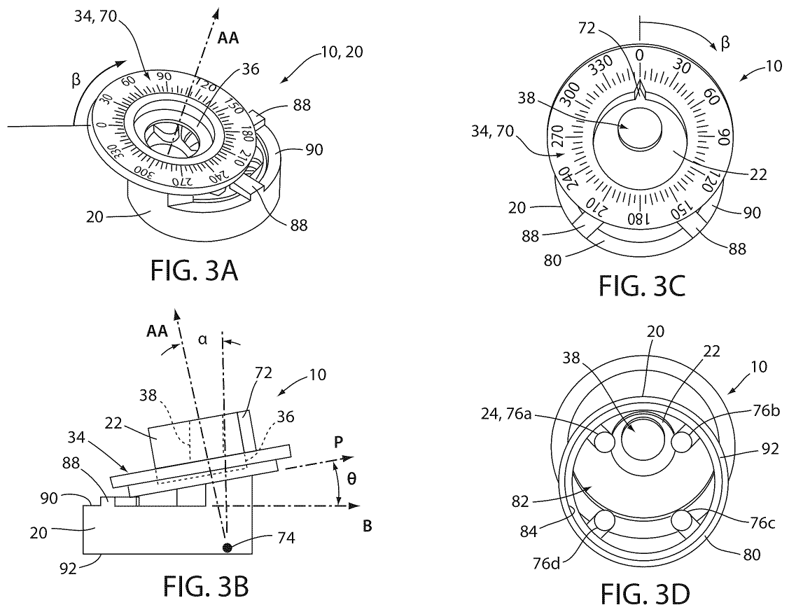

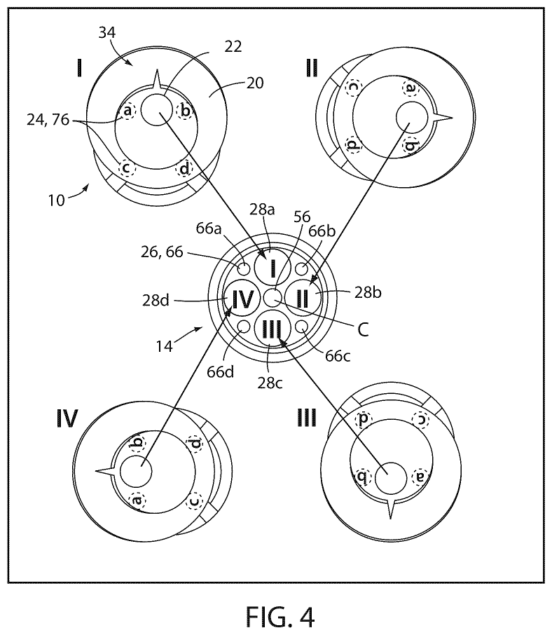

In FIGS. 1B and 1D, the alignment apparatus 10 is shown in connection with the base plate 14 in a third orientation III, which is further discussed in reference to FIG. 4. As shown, the coupling fixture 20 of the alignment apparatus 10 may be aligned with the base plate 14 by engaging the alignment features 24 of the coupling fixture 20 with the complementary mating features 26 of the base plate 14 (see FIGS. 2 and 3). With the coupling fixture 20 engaged with the base plate 14, the alignment axis AA of the alignment apparatus 10 may be centrally aligned with one of the fastener apertures 28 of the base plate 14. The engagement of the coupling fixture 20 to the base plate 14 may align a guide plane P of the alignment apparatus 10 at an incline angle θ relative to a base plane B of the base plate 14. The alignment axis AA may be oriented perpendicular to the incline angle θ. In this configuration, a neutral or central alignment of each of the fasteners 30 along the alignment axis AA may extend perpendicularly through the guide plane P oriented at the incline angle θ relative to the base plane B.

As later discussed in further detail in reference to FIGS. 3A-3D, the coupling fixture 20 may comprise an indicator surface 34, which may extend parallel to the guide plane P. The indicator surface 34 may be formed by a body of the coupling fixture 20 aligned with the guide plane P at the incline angle θ. A pocket 36 may be formed centrally through the indicator surface within the body of the coupling fixture 20. The pocket 36 may correspond to a cylindrical opening configured to receive a cylindrical profile shape of a guide insert 22. In this configuration, the guide insert 22 may engage the pocket 36, such that the guide insert 22 may rotate about a longitudinal axis of the cylindrical profile shape that extends along the alignment axis AA and perpendicular with the guide plane P. In various implementations, the incline angle θ may be implemented at various angles relative to the base plane B. For example, the incline angle θ demonstrated in the illustrated examples is approximately 12°. However, in various implementations, the incline angle θ may vary from approximately 1° to 45° depending on the application. The incline angle θ desired may be dependent on the relative proportions and specific geometry of the implant as well as the procedure or anatomy of the patient to which the implant is to be affixed. Accordingly, the incline angle θ and corresponding proportions of the alignment apparatus may vary based on the application.

With the guide insert 22 engaged in the pocket 36 of the coupling fixture 20, the guide insert 22 may be free to rotate about the alignment axis AA. As later discussed, a rotational angle β may define a rotational position of the guide inserts 22 in connection with the coupling fixture 20 about the alignment axis AA. As shown by the phantom lines extending through the guide insert 22 in FIGS. 1B and 1D, a guide aperture 38 may extend through the guide insert 22. The guide aperture 38 may correspond to a cylindrical opening configured to receive a surgical tool 40 (e.g., a drill or driver) through a body of the guide insert 22. The guide aperture 38 may extend at an insert angle α or second angle which may be defined in reference to the alignment axis AA. In this configuration, the rotational angle β of the guide insert 22 may adjust the rotational orientation or direction of the insert angle α about the alignment axis AA. Accordingly, the trajectory 32 of the surgical tool 40 may be adjusted by rotating the guide insert to a selected rotational angle β at a selected insert angle α of the guide insert 22. For clarity, an exemplary rotation of the driver 40 is demonstrated by an arrow 42. As provided in the following detailed examples, the alignment orientation I, II, III, IV of the coupling fixture 20 may be adjusted to selectively align the trajectory 32 of each of the fasteners 30. As discussed further in the following examples, the alignment apparatus 10 may provide for the selective alignment of the trajectories 32 to efficiently and accurately orient each of the fasteners 30 and corresponding pilot holes with the anatomy of a patient.

Referring now to FIG. 2, the base plate 14 of the implant 12 is demonstrated in further detail. As shown, the base plate 14 may comprise a hub portion 50 forming a cylindrical perimeter wall 52 and a flange 54. A central aperture 56 extends along a central axis C of the base plate 14. The central aperture 56 may provide for alignment of the base plate 14 along a guide pin during a surgical procedure. The fastener apertures 28 may be radially distributed about the central axis C and may extend proximate to the cylindrical perimeter wall 52. In the example shown, the complementary mating features 26 may correspond to receiving apertures (e.g., circular or round openings) configured to receive the alignment features 24 in the form of alignment posts or protrusions. Additionally, the complementary mating features 26 may also be radially distributed about the central axis C interposed between each of the fastener apertures 28. In this configuration, the complementary mating features 26 and the fastener apertures 28 may correspond to radially symmetric openings distributed evenly about the central axis C.

For clarity, the illustrated example shown in FIG. 2 demonstrates the base plate 14 comprising a first aperture 28a, a second aperture 28b, a third aperture 28c, and a fourth aperture 28d. The base plate 14 may be connected with the glenoid cavity via a plurality of fasteners 30 comprising a first fastener 30a, a second fastener 30b, a third fastener 30c, and a fourth fastener 30d. The fasteners 30a, 30b, 30c, 30d may aligned with each of the respective, enumerated apertures 28a, 28b, 28c, 28d. Though specific numeric designations are referenced to identify the apertures 28, fasteners 30, and various aspects of the disclosure, such designations are provided for clarity to distinguish among similar elements in the provided examples. Accordingly, the number of elements described in the various examples provided shall not be considered limiting to the scope of the disclosure.

As further demonstrated in FIGS. 1B and 2, the base plate 14 may further comprise a cowl 58, which may be configured to extend about the base plate 14 enclosing an exposed portion of the glenoid cavity 16. Exemplary fasteners 30 are demonstrated along simulated trajectories 32 that may be aligned with the aid of the alignment apparatus 10 as discussed herein. Additionally, as shown, a perimeter or auxiliary fastener 60 may engage an aperture formed through the cowl 58, which may serve to further secure the base plate 14 to the anatomy of the patient. Once the base plate 14 is secured to the patient, a glenosphere (not shown) or additional components of the implant 12 may be affixed to complete the assembly of the joint replacement.

The arrangement of the complementary mating features 26 of the alignment features 24 may be configured to correspond to the number of fastener apertures 28 radially distributed about the central axis C. For example, the base plate 14 may comprise a plurality of mating or alignment apertures 66 forming four alignment apertures 66a, 66b, 66c, 66d. As shown, the alignment apertures 66a, 66b, 66c, 66d may be radially distributed about the central axis C of the base plate 14 between neighboring fastener apertures 28. More specifically, a first alignment aperture 66a may be located between the fourth fastener aperture 28d and the first fastener aperture 28a. Similarly, a second alignment aperture 66b may be located between the first and second fastener apertures 28a, 28b; and a third alignment aperture 66c may be located between the second and third fastener apertures 28b, 28c. Additionally, a fourth alignment aperture 66d may be located between the third and fourth fastener apertures 28c, 28d. In this configuration, the number of alignment features 24 and corresponding complementary mating features 26 may directly correspond to the number of fastener apertures 28. As later discussed in reference to FIG. 4, the numeric correspondence and even radial spacing of the alignment features 24 and the fastener apertures 28 may provide for a rotational symmetry and corresponding rotational mating orientations I, II, III, and IV between the coupling fixture 20 and the base plate 14 of the implant 12. As previously mentioned, though the number of corresponding features discussed in the provided examples is four, it shall be understood that the number of complementary mating features 26 and fastener apertures 28 may include various numbers of corresponding features (e.g., two apertures each, three apertures each, five apertures each, etc.).

Referring now to FIGS. 3A-3D, the alignment apparatus 10 is demonstrated in a variety of projected views to demonstrate various features. Referring first to FIG. 3A, the coupling fixture 20 is shown without the guide insert 22 installed. As shown, the pocket 36 may provide for a cylindrical opening that may receive a complementary cylindrical profile shape of the guide insert 22. The indicator surface 34 may extend about a perimeter of the pocket 36 and may include an angular indicia 70, which may correspond to incremental markings and/or characters arranged to form a protractor or cylindrical dial. The angular indicia 70 may provide for a visual indication of a direction of the insert angle α as indicated by the rotational angle β about the alignment axis AA. As demonstrated in FIG. 3C, the guide insert 22 may also comprise an alignment indicator 72, which may correspond to a marking, arrow, pointer, protrusion, etc. In operation, the alignment indicator 72 may provide a visual indication of the rotational angle β of the insert angle α about the alignment axis AA relative to the angular indicia 70.

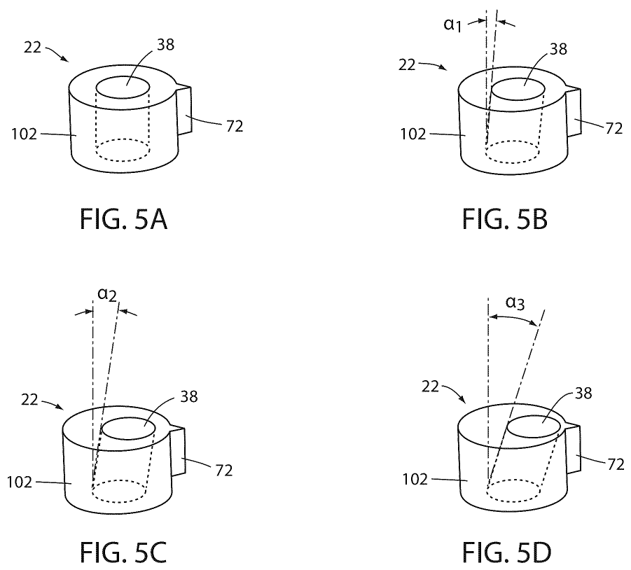

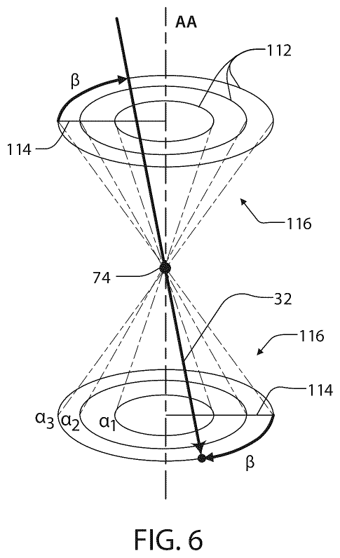

Referring now more specifically to FIG. 3B, the guide angle θ of the guide plane P is clearly shown in reference to the base plane B. As previously discussed, the base plane B may be aligned perpendicular to the central axis C of the base plate 14 and parallel to a mating surface designated by the convergence point 74. FIG. 3B further demonstrates the insert angle α of the guide aperture 38 relative to the alignment axis AA. In various implementations, the insert angle α may be varied by utilizing a plurality of interchangeable guide inserts 22. As further discussed in reference to FIG. 5, each of the interchangeable guide inserts 22 may have different insert angles α. Accordingly, the insert angle α may be adjusted by selecting a corresponding interchangeable guide insert 22 to define the magnitude of the insert angle α and the associated trajectory 32 relative to the alignment axis AA. Though the insert angles α and corresponding cylindrical opening formed by the guide aperture 38 may vary among the interchangeable guide inserts 22, the alignment axis AA and the insert angle α may consistently converge at or approximate to the convergence point 74, which may correspond to a seating position of the corresponding fastener 30. In this configuration, the relationship between the insert angle α of the guide insert 22 and the guide angle θ of the guide plane P may provide for the guide insert 22 to rotate about a tilted axis that may vary based on the selected insert angle α while consistently intersecting with the convergence point 74. Further discussion regarding the rotation of the guide insert 22 is provided in reference to FIG. 6. In various implementations, the alignment apparatus 10 may provide for each of the fasteners 30 to be aligned with various angular trajectories 32 while maintaining convergence with a seating position formed by each of the fastener apertures 28.

FIGS. 3C and 3D demonstrate top and bottom views of the alignment apparatus 10, respectively. As shown in FIG. 3C, the guide inserts 22 may rotate within the pocket 36 formed within the indicator surface 34. The alignment indicator 72 may identify the rotational angle β of the guide insert 22 relative to the coupling fixture 20. In the bottom view demonstrated in FIG. 3D, the alignment features 24 are shown as cylindrical protrusions 76 that extend from a perimeter collar or collar 80 into a hub alignment opening 82. As shown, an interior perimeter wall 84 of the collar 80 may be configured to receive the hub portion 50 and align with the cylindrical perimeter wall 52 of the base plate 14, such that the central axis C of the base plate 14 is aligned centrally within the collar 80. In this configuration, the rotational orientation of the cylindrical protrusions 76 forming the complementary mating features 26 may be aligned about the central axis C. The hub alignment opening 82 of the collar 80 may rotate about the cylindrical perimeter wall 52 while maintaining an alignment of the alignment features 24 about the central axis C. With the alignment features 24 aligned radially about the central axis C, each of the rotational orientations I, II, III, IV may be engaged by rotating the coupling fixture 20 relative to the base plate 14 until a sliding engagement with the complementary mating features 26 is aligned. Further details regarding the selective alignment of the coupling fixture 20 with the base plate 14 are described in reference to FIG. 4.

Referring to FIGS. 2 and 3D, the protrusions 76 may be the same in number or have a numeric correspondence (e.g., the same number or a factor of) the number of alignment apertures 66. For example, in the first orientation I discussed later in reference to FIG. 4, a first protrusion 76a may align with the first alignment aperture 66a, and a second protrusion 76b may align with the second alignment aperture 66b. Further, a third protrusion 76c may align with the third alignment aperture 66c, and a fourth protrusion 76d may align with the fourth alignment aperture 66d. Each of the orientations I, II, III, IV may adjust the alignment of the protrusions 76 with the apertures 66 in successive rotations about the central axis C to align the alignment axis AA with the associated fastener aperture 28. The alignment orientations I, II, III, IV and mating arrangements of the alignment features 24 to the complementary mating features 26 are discussed and demonstrated in further detail in FIG. 4.

Finally, as shown in FIGS. 3A, 3B, 3C, and 3D, the cylindrical protrusions 76 may be supported by mounting tabs or support brackets 88 that may extend into the hub alignment opening 82 from a top surface 90 of the collar 80. In this configuration, the cylindrical protrusions 76 forming the alignment features 24 of the coupling fixture 20 may engage the complementary mating features 26 or alignment apertures 66 formed in the cylindrical perimeter wall 52 of the hub portion 50. A bottom surface 92 of the collar 80 may further provide for a positive stop that engages the flange 54 of the base plate 14 in an assembled configuration. The sliding engagement of each alignment features 24 with the base plate 14 may provide for simplified and intuitive transitions to sequentially affix each of the fasteners 30 within the fastener apertures 28.

Referring to FIG. 4, a simplified top view of the alignment apparatus is shown in a first orientation I, a second orientation II, a third orientation III, and a fourth orientation IV. Each of the orientations I, II, III, IV of the alignment apparatus 10 are shown with an arrow indicating the fastener aperture 28 of the base plate 14 aligned with the alignment axis AA in the corresponding orientation. The cylindrical protrusions 76 forming the alignment features 24 of the coupling fixture 20 are labeled a, b, c, d to clearly designate the relative orientation of the coupling fixture 20 to the base plate 14. As shown, the rotational symmetry of the alignment features 24 about the central axis C of the base plate 14 may provide for the guide aperture 38 and the alignment axis AA to align with each of the fastener apertures 28 by rotating the alignment apparatus 10 by 90° about the central axis C relative to the base plate 14.

In operation, the alignment apparatus 10 may be installed in connection with the base plate 14 by aligning the cylindrical protrusions 76 with the corresponding complementary mating features 26 exemplified by the alignment apertures 66 of base plate 14. The orientation of the alignment apparatus 10 may then be sequentially changed among the orientations I, II, III, and IV by sliding the alignment apparatus 10 away from the base plate 14 (out of the page as depicted) along the cylindrical protrusions 76 and rotating the alignment apparatus 10 by 90° in the example shown. The next corresponding orientation of the alignment apparatus 10 may then be aligned by engaging the cylindrical protrusions 76 of the alignment apparatus 10 with the complementary mating features 26 of the base plate 14 in the newly rotated orientation. In this way, the alignment axis AA of the alignment apparatus 10 may be sequentially aligned with the base plate 14 in each of the orientations I, II, III, and IV.

Though discussed specifically in reference to four fastener apertures 28 and four corresponding cylindrical protrusions 76, the alignment apparatus 10 may similarly be implemented with varying numbers of fasteners and alignment features. For example, the four cylindrical protrusions 76 demonstrated provide for a fourth order rotational symmetry that repeats four times per revolution or every 90° rotation of the alignment apparatus 10. Accordingly, it shall be understood that a similar rotational symmetry may be implemented for the alignment apparatus 10 for two apertures, three apertures, five apertures, etc. For example, in the case of three fastener apertures, the radial spacing among the alignment features 24 and the fastener apertures 28 may be 120° about the central axis C. In this configuration, the alignment apparatus 10 may provide for a third order rotational symmetry. For two fasteners 30, the fastener apertures 28 and alignment features 24 may be spaced-apart by 180°. For five fasteners 30, the corresponding fastener apertures 28 and alignment features 24 may be repeated every 72° about the central axis C providing for a fifth order rotational symmetry. Accordingly, the rotational orientations of the alignment apparatus 10 may be implemented in a variety of configurations that may correspond to the number of fasteners 30 equally spaced about a central axis C of the base plate 14.

Referring to FIGS. 5A-5D, examples of the interchangeable guide inserts 22 are shown demonstrating different insert angles α. Each of the guide inserts 22 may include the guide aperture 38 formed at a different insert angle α through a cylindrical body 102 forming the guide insert 22. As demonstrated in FIG. 5A, the insert angle α is 0°, such that the trajectory 32 may be aligned with the alignment axis AA. In each of FIGS. 5B-5D, the insert angle α is gradually increased demonstrating a relative insert angle α of approximately 0.5° in FIG. 5B, 3° in FIG. 5C, and 6° in FIG. 5D. Though referred to in reference to specific angular examples as shown in FIGS. 5A-5D, the insert angle α may generally vary from approximately 0.1° to 20°. In some embodiments, the insert angle α may vary from 0.5° to 10°. In various implementations, the interchangeable guide inserts 22 may be configured to provide specific insert angles α over a desired range and with a designated step size or interval spacing among the corresponding insert angles α. Accordingly, the specific angle of the guide aperture 38 of the guide insert 22 may be implemented over a wide range of angles to suit a variety of applications.

In order to better understand the complex nature of the angular orientation of the trajectories 32 that may be provided by the alignment apparatus 10, a simplified diagram is shown in FIG. 6 demonstrating a plurality of the trajectories 32 aligned by the guide insert 22. The trajectories 32 are shown in relation to the convergence point 74 and may vary based on variations in the insert angle α and the rotation angle β. As shown, the alignment axis AA is represented vertically rather than angled at the guide angle β along the guide plane P. A plurality of ellipses 112 are shown representing a projected view of circular paths of each of the trajectories 32 aligned through the guide aperture 38 of the guide insert 22. The insert angles α₁, α₂, and α₃ demonstrate increasing angles and corresponding increasing divergence of the trajectories 32 away from the alignment axis AA. As previously discussed, the severity or magnitude of the insert angle α may be determined based on the interchangeable guide insert 22 selected.

The specific example shown for the trajectory 32 for the guide inserts 22 has the third insert angle α₃. As shown, the rotational angle β is rotated approximately 60° clockwise from a reference marker 114. The resulting trajectory 32 passes through the convergence point 74 to an equal and opposite position demonstrated on a conical shape 116 representative of the trajectory 32 rotated about the rotational angle β. As shown, the conical shape 116 may provide a visual reference identifying the trajectory 32 between the ellipses 112. In the example shown, the conical shape 116 corresponds to the third insert angle α₃ rotated 360° about the alignment axis AA. In addition to the selective alignment of the trajectories 32 along the insert angle α and rotational angle β as demonstrated in FIG. 6, the trajectories 32 are further defined by the guide angle θ and the corresponding orientation of the alignment apparatus 10 in connection with the base plate 14 in each of the orientations I, II, III, and IV as previously discussed in reference to FIG. 4.

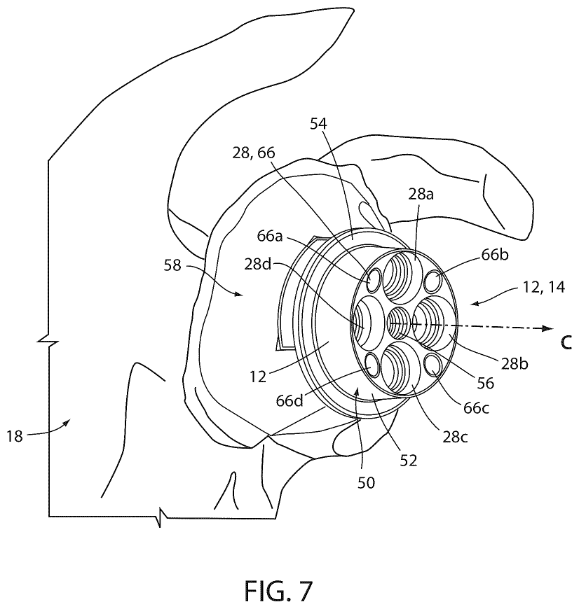

Referring now to FIGS. 7, 8A-8B, and 9A-9C, an exemplary method of implementing the alignment apparatus 10 is described in further detail. The example provided corresponds to a surgical procedure for securing the base plate 14 to a glenoid cavity 16 of a patient. As demonstrated in FIG. 7, the alignment apparatus 10 may be utilized to clear or drill pilot holes through the fastener apertures 28 of the base plate 14 once the base plate 14 is aligned within the glenoid cavity 16. Additionally, the alignment apparatus 10 may similarly be implemented to guide the trajectories 32 of the fasteners 30 to seated positions within the fastener apertures 28 of the base plate 14. In this way, the alignment apparatus 10 may provide for the selective alignment of each of a plurality of fasteners 30 according to trajectories 32 per a surgical plan of a patient.

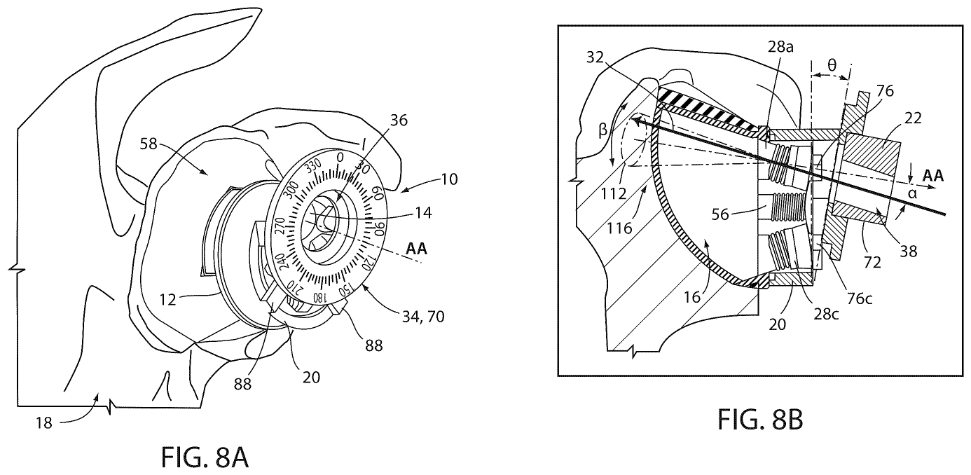

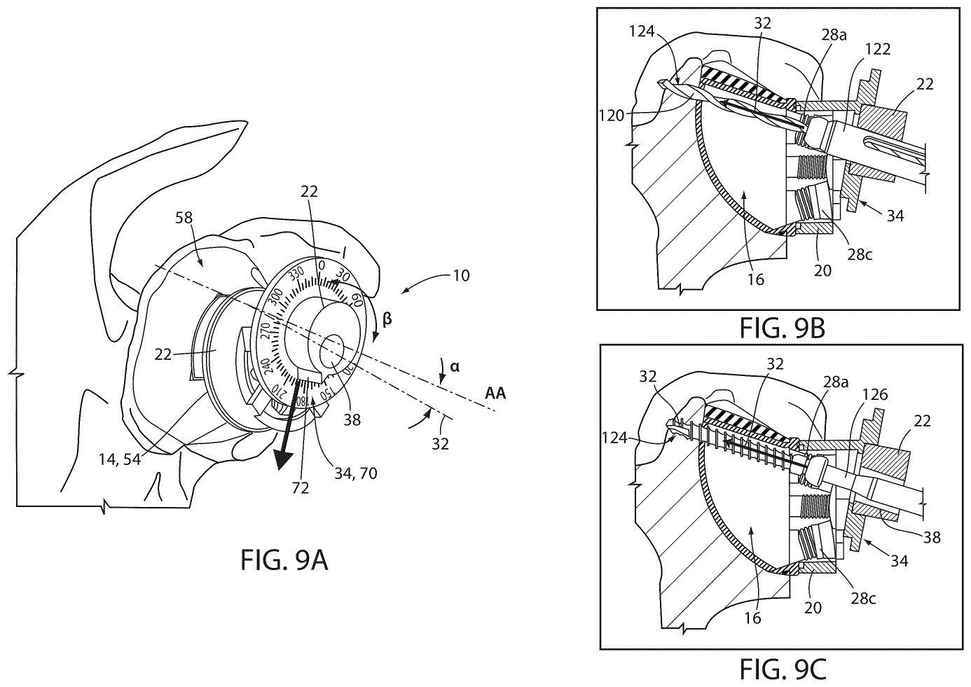

Referring now to FIGS. 7 and 8A, the alignment features 24 (e.g., the cylindrical protrusions 76) of the alignment apparatus 10 may slide into and engage the complementary mating features 26 (e.g., alignment apertures 66) to align the coupling fixture 20 with the base plate 14 in each of the alignment orientations I, II, III, and IV. As demonstrated in FIG. 8A, the alignment apparatus 10 is demonstrated in connection with the base plate 14 in the first orientation I. In the first orientation I, the coupling fixture 20 may position the alignment axis AA aligned through the first fastener aperture 28a. Following the positioning and installation of the first fastener 30a with the first fastener aperture 28a in the first orientation I, each of a second fastener 30b, the third fastener 30c, and the fourth fastener 30d may sequentially be aligned and fastened to the base plate 14. More specifically, the second fastener aperture 28b may be aligned in the second orientation II, the third fastener aperture 28c in the third orientation III, and the fourth fastener aperture 28d in the fourth orientation IV. In this way, each of the fasteners 30 may be sequentially positioned and affixed to the corresponding fastener apertures 28 by repeating the procedures discussed in reference to FIGS. 8A-8B and 9A-9C.

Referring to FIG. 8A, once the coupling fixture 20 is aligned with the base plate 14 via the alignment features 24 and the complementary mating features 26, the coupling fixture 20 may be aligned with the selected fastener aperture 28 with the alignment axis angled at the guide angle θ. The guide insert 22 may then be selected from the plurality of interchangeable guide inserts 22 based on the desired insert angle α. The selected guide insert 22 may be seated in the pocket 36 formed in the indicator surface 34. As shown in FIGS. 8B and 9A, the trajectory 32 of a pilot hole and fastener 30 may then be oriented at the selected insert angle α based on the rotational angle β of the guide insert 22 about the alignment axis AA. As shown in FIG. 9A, the alignment indicator 72 is aligned with the angular indicia indicating that the rotational angle β is approximately 190°.

As shown in FIGS. 9A and 9B, the alignment apparatus 10 is shown positioned in the desired alignment orientation (e.g., orientation I) with the guide insert 22 selected for the desired insert angle α and oriented at the desired rotational angle β. In this configuration, the trajectory 32 of the alignment device 10 may be set based on a surgical plan for a patient. Once the trajectory 32 of the alignment apparatus 10 is set through the guide aperture 38, a physician or surgeon may insert a cutting tool 120 (e.g., a drill, boring tool, etc.) into the guide aperture 38 to align the path of the cutting tool 120 with the trajectory 32. As demonstrated in FIG. 9B, the cutting tool 120 engages the guide aperture 38 of the guide insert 22 via a sleeve 122 interposed between the guide insert 22 and the cutting tool 120. In this configuration, the path of the cutting tool 120 may be aligned with the trajectory 32 while preserving the integrity of the alignment apparatus 10 from a rotating motion of the cutting tool 120. By extending the cutting tool 120 along the trajectory 32, a pilot hole 124 may be formed in bone or tissue of the patient to prepare and clear an opening for positioning and attaching the fastener 30.

As demonstrated in FIG. 9C, once the pilot hole 124 is formed, the position of the alignment apparatus 10 may be maintained to align the fastener 30 with the trajectory 32 through the guide aperture 38. As shown, a driver 126 (e.g., a screwdriver, screw gun, etc.) may be implemented to engage the fastener 30 via the guide aperture 38 and drive the fastener 30 to a seated position within the fastener aperture 28 of the base plate 14. As previously discussed, once the first fastener 30a is connected within the first fastener aperture 28a associated with the first orientation I, the process described in reference to FIGS. 8A-8B and 9A-9C may be repeated until each of the fasteners 30 are secured within the corresponding fastener apertures 28. Accordingly, the alignment apparatus 10 may provide for the simple and accurate alignment of each of the trajectories 32 of the fasteners 30 to secure the base plate 14 to the anatomy of a patient.

The flexibility and simplicity of operation of the alignment apparatus 10 may provide significant benefits to improve the associated surgical methods by providing both flexibility in adjusting the trajectories 32 of the fasteners 30 while also maintaining efficiency and accuracy. Accordingly, the alignment apparatus 10 may provide surgeons with the flexibly adjust a surgical plan for a patient during an operation without requiring complex operation. In this way, the methods associated with the use of the alignment apparatus 10 may limit surgery times while improving the accuracy of the alignment of the fasteners 30 with the desired trajectories 32 to improve patient outcomes.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents.

1. An alignment apparatus for an orthopedic implant, the apparatus comprising:
a coupling fixture comprising a plurality of alignment features that align with and engage complementary features of the orthopedic implant, wherein the alignment features orient an alignment axis of the alignment apparatus with at least one fastener aperture of the orthopedic implant; and
a guide insert comprising a guide aperture through which the alignment axis passes, wherein the guide aperture is configured to selectively align a tool or fastener with a plurality of trajectories through the at least one fastener aperture.

2. The alignment apparatus according to claim 1, wherein the at least one fastener aperture of the implant comprises a plurality of fastener apertures, and wherein the alignment features engage the complementary features in a plurality of rotational orientations relative to the orthopedic implant.

3. The alignment apparatus according to claim 2, wherein the plurality of rotational orientations selectively align the alignment axis of the coupling fixture with each of the plurality of fastener apertures.

4. The alignment apparatus according to claim 2, wherein the coupling fixture comprises a collar that receives the orthopedic implant and aligns a central axis of the implant with the alignment features.

5. The alignment apparatus according to claim 1, wherein the alignment features comprise a plurality of alignment pins that engage a plurality of coupling apertures of the orthopedic implant.

6. The alignment apparatus according to claim 1, wherein the coupling fixture aligns the alignment axis at a first angle relative to a mounting surface of the orthopedic implant.

7. The alignment apparatus according to claim 6, wherein the first angle is between 1° and 45°.

8. The alignment apparatus according to claim 1, wherein the guide insert engages the coupling fixture along a guide plane defined by a first angle and perpendicular to the alignment axis.

9. The alignment apparatus according to claim 8, wherein the guide insert is rotationally constrained to rotate about the alignment axis in an assembled configuration engaged with the coupling fixture.

10. The alignment apparatus according to claim 8, wherein the guide aperture of the guide insert is angled at a second angle relative to the alignment axis.

11. The alignment apparatus according to claim 10, wherein the second angle of the plurality of guide inserts varies from 0.1° to 15°.

12. The alignment apparatus according to claim 10, wherein the first angle and the second angle define a compound angle defining a path of the trajectories through the at least one fastener aperture.

13. The alignment apparatus according to claim 12, wherein a rotation of the guide insert relative to the coupling fixture selectively aligns the guide aperture with the trajectories of the tool or fastener.

14. The alignment apparatus according to claim 10, wherein the guide insert is an interchangeable component comprising a plurality of guide inserts, wherein the second angle of each of the plurality of guide inserts is different, such that the second angle is selected based on the guide insert selected.

15. The alignment apparatus according to claim 1, wherein the guide insert corresponds to a cylindrical dial that engages a pocket of the coupling fixture.

16. The alignment apparatus according to claim 15, wherein the coupling fixture comprises an indicator surface extending about the pocket of the coupling fixture.

17. The alignment apparatus according to claim 16, wherein the indicator surface comprises an angle indicia and the guide insert comprises an alignment indicator, and wherein a rotational angle of the guide insert relative to the coupling fixture is identified by an orientation of the alignment indicator with the angle indicia.

18. The alignment apparatus according to claim 1, wherein the orthopedic implant is a glenoid implant.

19. A method for aligning a fastener for an orthopedic implant, the method comprising:
aligning an alignment axis of a coupling fixture with a first fastener aperture of a plurality fastener apertures of the orthopedic implant by engaging a plurality of alignment features with a plurality of complementary features of the orthopedic implant in a first orientation;
removing the coupling fixture from engagement with the orthopedic implant;
rotating the coupling fixture relative to the orthopedic implant; and
aligning the coupling fixture with a second fastener aperture of a plurality fastener apertures of the orthopedic implant by engaging the plurality of alignment features with the plurality of complementary features of the orthopedic implant in a second orientation.

20. The method according to claim 19, wherein the coupling fixture engages the orthopedic implant by sliding the alignment features linearly along the complementary features of the orthopedic implant.

21. The method according to claim 19, wherein the rotation of the coupling fixture is about a central axis of the orthopedic implant and aligns the plurality of alignment features with the plurality of complementary features of the orthopedic implant.

22. The method according to claim 21, wherein the alignment features and the fastener apertures are evenly distributed about the central axis.

23. The method according to claim 19, further comprising:
selectively engaging a guide insert comprising a guide aperture with the coupling fixture, wherein the engagement between the guide insert and the coupling fixture constrains a rotation of the guide insert to rotate about the alignment axis.

24. The method according to claim 23, wherein the guide aperture is angled at a first angle relative to the alignment axis, wherein the rotation of the guide insert about the alignment axis adjusts a trajectory angle of the guide aperture relative to the alignment axis; and
further comprising aligning a tool or fastener through the guide aperture at a rotational angle about the alignment axis, thereby defining a trajectory of the tool or fastener.

25. An alignment apparatus for an orthopedic implant, the apparatus comprising:
a coupling fixture comprising a plurality of alignment features that align with complementary features of the orthopedic implant, wherein the alignment features orient an alignment axis of the alignment apparatus with at least one fastener aperture of the orthopedic implant; and
a guide insert that engages the coupling fixture along a guide plane defined by a first angle perpendicular to the alignment axis, wherein a rotation of the guide insert relative to the coupling fixture is constrained to rotate about the alignment axis in an assembled configuration, and wherein the guide insert comprises a guide aperture angled at a second angle relative to the alignment axis, wherein the rotation of the guide insert relative to the coupling fixture selectively aligns the guide aperture with a plurality of trajectories of a tool or a fastener.
